(19) United States
(12) Patent Application Publication
(10) Pub. No.: US 2010/0114141 A1
(43) Pub. Date: May 6, 2010

(54) OPTIMIZING THE OPERATION OF AN INTRA-GASTRIC SATIETY CREATION DEVICE

(76) Inventors: Thomas E. ALBRECHT, Cincinnati, OH (US); Jason L. HARRIS, Mason, OH (US); Mark S. ORTIZ, Milford, OH (US); Michael J. STOKES, Cincinnati, OH (US); Mark S. ZEINER, Mason, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/261,086

(22) Filed: Oct. 30, 2008

(51) Int. Cl. A61M 29/02; A61B 5/00

(52) U.S. Cl. 606/191; 600/561

(57) ABSTRACT

A method for determining an optimum control parameter of a distension system for causing distension in a stomach. The method includes the step of providing an implantable distension system for causing distension in a stomach, the system including an adjustable distension device configured to form a distension in a stomach. The method also involves adjusting the distension device, and determining the value of a control parameter of the distension system. The method also involves repeating the steps of adjusting the distension device and determining the value of the control parameter until the control parameter is substantially convergent as a function of time.

This case is related to the following commonly assigned and concurrently filed U.S. Applications, all of which are hereby incorporated herein by reference:
U.S. Ser. No. ______ (Attorney Docket Number END6514USNP) titled DEVICES and METHODS FOR ADJUSTING A SATIATION AND SATIETY-INDUCING IMPLANTED DEVICE; U.S. Ser. No. ______ (Attorney Docket Number END6515USNP) titled Sensor Trigger; U.S. Ser. No. ______ (Attorney Docket Number END6516USNP) titled AUTOMATICALLY ADJUSTING INTRA-GASTRIC SATIATION AND SATIETY CREATION DEVICE; U.S. Ser. No. ______ (Attorney Docket Number END6517USNP) titled OPTIMIZING THE OPERATION OF AN INTRA-GASTRIC SATIETY CREATION DEVICE; U.S. Ser. No. ______ (Attorney Docket Number END6518USNP) titled POWERING IMPLANTABLE DISTENSION SYSTEMS USING INTERNAL ENERGY HARVESTING MEANS; U.S. Ser. No. ______ (Attorney Docket Number END6519USNP) titled WEARABLE ELEMENTS FOR INTRA-GASTRIC SATIETY CREATION SYSTEMS; U.S. Ser. No. ______ (Attorney Docket Number END6520USNP) titled INTRA-GASTRIC SATIETY CREATION DEVICE WITH DATA HANDLING DEVICES AND METHODS; U.S. Ser. No. ______ (Attorney Docket Number END6521USNP) titled GUI FOR AN IMPLANTABLE DISTENSION DEVICE AND A DATA LOGGER; U.S. Ser. No. ______ (Attorney Docket Number END6522USNP) titled METHODS AND DEVICES FOR FIXING ANTENNA ORIENTATION IN AN INTRA-GASTRIC SATIETY CREATION SYSTEM; U.S. Ser. No. ______ (Attorney Docket Number END6523USNP) titled METHODS AND DEVICES FOR PREDICTING INTRA-GASTRIC SATIETY CREATION DEVICE SYSTEM PERFORMANCE; U.S. Ser. No. ______ (Attorney Docket Number END6524USNP) titled CONSTANT FORCE MECHANISMS for Regulating Distension Devices; U.S. Ser. No. ______ (Attorney Docket Number END6525USNP) titled A METHOD OF REMOTELY ADJUSTING A SATIATION AND SATIETY-INDUCING IMPLANTED DEVICE.

### FIELD OF THE INVENTION

The present invention relates to methods and devices for optimizing the operation of a gastric distension system.

### BACKGROUND OF THE INVENTION

Obesity is becoming a growing concern, particularly in the United States, as the number of obese people continues to increase, and more is learned about the negative health effects of obesity. Morbid obesity, in which a person is 100 pounds or more over ideal body weight, in particular poses significant risks for severe health problems. Accordingly, a great deal of attention is being focused on treating obese patients. One proposed method of treating morbid obesity has been to place a distension device, such as a, spring loaded coil inside the stomach. Examples of satiation and satiety inducing gastric implants, optimal design features, as well as methods for installing and removing them are described in commonly owned and pending U.S. patent application Ser. No. 11/469,564, filed Sep. 1, 2006, and pending U.S. patent application Ser. No. 11/469,562, filed Sep. 1, 2006, which are hereby incorporated herein by reference in their entirety. One effect of the distension device is to more rapidly induce feelings of satiation defined herein as achieving a level of fullness during a meal that helps regulate the amount of food consumed. Another effect of the distension device is to prolong the effect of satiety which is defined herein as delaying the onset of hunger after a meal which in turn regulates the frequency of eating. By way of a non-limiting list of examples, positive impacts on satiation and satiety may be achieved by an intragastric distension device through one or more of the following mechanisms: reduction of stomach capacity, rapid engagement of stretch receptors, alterations in gastric motility, pressure induced alteration in gut hormone levels, and alterations to the flow of food either into or out of the stomach.

With each of the above-described stomach distension devices, safe, effective treatment requires that the device be regularly monitored and adjusted to vary the degree of distension applied to the stomach.

During these adjustments, it may be difficult to determine how the adjustment is proceeding, and whether the adjustment will have the intended effect. In an attempt to determine the efficacy of an adjustment, some physicians may utilize fluoroscopy with a Barium swallow as the adjustment is being performed. However, fluoroscopy is both expensive and undesirable due to the radiation doses incurred by both the physician and patient. A physician may simply adopt a “try as you go” method based upon their prior experience, and the results of an adjustment may not be discovered until hours or days later, when the patient experiences a too much distension to the stomach cavity, or the distension device induces erosion of the stomach tissue due to excessive interface pressures against the tissue.

It is often desirable to collect data concerning the operation of the distension system as well as concerning the physiological characteristics of the patient. A distension system may be equipped with a variety of sensors that can be configured to collect and transmit data that is useful for adjustment, diagnostic, monitoring, and other purposes. However, even these sensor equipped distension systems would require the physician to perform a series of adjustments to the system that often involve trial and error.

Accordingly, methods and devices are provided for use with a gastric distension system, and in particular for optimizing the operation of a distension system.

### BRIEF DESCRIPTION OF THE DRAWINGS

The invention will be more fully understood from the following detailed description taken in conjunction with the accompanying drawings, in which:

FIG. 1A is a perspective view of one embodiment of a food intake distension system;

FIG. 1B is side view of one embodiment of a distension system;

FIG. 2A is a cross sectional view of the gastric coil of the distension system shown in FIG. 1B;

FIG. 2B is a perspective view of the gastric coil shown in FIG. 2A as applied to the stomach of a patient;

FIG. 3 is a perspective view of the fluid injection port of the distension system shown in FIG. 1B;

FIG. 4 is a side view of another embodiment of a distension system;

FIG. 5 is a perspective view of the sensor housing shown in FIG. 1A;

FIG. 6 is a schematic of an embodiment of a variable resistance circuit for the pressure sensor of FIG. 5;

FIG. 7 is a block diagram of one embodiment of a pressure management system for use in conjunction with the distension system shown in FIG. 4;

FIG. 8 is a flow diagram of one embodiment of a method for optimizing the operation of a distension system for causing distension in a stomach;

FIG. 9 is a flow diagram of one embodiment of a method for determining an optimum control parameter of a distension system for causing distension in a stomach;

FIG. 10 is a flow diagram of one embodiment of a method for optimizing the operation of a distension system for causing distension in a stomach;

FIG. 11 is a flow diagram of one embodiment of a method for determining an optimum control parameter of a distension system for causing distension in a stomach;

FIG. 12 is a flow diagram of one embodiment of a method for returning a control parameter of a distension system for causing distension in a stomach to an optimum value;

FIG. 13A is a graphical representation of the value of a control parameter of a distension system as a function of time;

FIG. 13B is a graphical representation of the value of a result parameter of the distension system of FIG. 13A as a function of time;

FIG. 14A is a graphical representation of the value of a control parameter of a distension system as a function of time; and

FIG. 14B is a graphical representation of the value of a result parameter of the distension system of FIG. 14A as a function of time.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides devices and methods for optimizing the operation of a distension system for causing distension in a stomach. In one exemplary embodiment, a method for optimizing the operation of a gastric distension system includes providing an implantable distension system for causing distension in a stomach, determining an optimum value of a control parameter of the distension system, and maintaining the control parameter at the optimum value such that a result parameter of the distension system has a substantial convergence as a function of time. The implantable distension system of the method can have a variety of configurations. In general, the distension system can include an adjustable distension device that is configured to form a distension in a stomach. Exemplary non-limiting examples of adjustable implantable distension devices (e.g., satiation and satiety inducing gastric implants), optimal design features, as well as methods for installing and removing them are described in commonly owned and pending U.S. patent application Ser. No. ______, filed on even date herewith and entitled “Devices and Methods for Adjusting a Satiation and Satiety-Inducing Implanted Device” [Atty. Docket No. END6514USNP], which is hereby incorporated herein by reference in its entirety.

Determining an optimum value of a control parameter can generally include adjusting the distension device, determining the value of the control parameter to be optimized, and repeating the steps of adjusting the distension device and determining the value of the control parameter until the control parameter is substantially convergent as a function of time (i.e., until the value of the control parameter substantially converges on a value over time). In one exemplary embodiment, determining the optimum value of a control parameter can further include detecting a value of the control parameter and comparing the detected value to a previously determined value of the control parameter.

If the detected value of the control parameter and the previously determined value of the control parameter are not substantially equal, the distension device can be adjusted. A number of factors can affect the adjustment of the coil. For example, the operating parameter chosen by the physician to be the control parameter, the measured value of the control parameter, and how the control parameter is measured can all influence the adjustment of the coil. In one exemplary embodiment, if the detected measurement of the control parameter is less than the previously determined value of the control parameter, the distension device can be expanded, causing more distension. The distension device can generally be expanded by increasing the pressure within the distension system. Alternatively, in another exemplary embodiment, if the detected measurement of the control parameter is greater than the previously determined value of the control parameter, the distension device can be reduced. In one embodiment, the distension device can be reduced by decreasing the pressure within the distension system. As indicated above, several factors can affect the adjustment of the coil. Thus, a detected value of the control parameter that is greater than a previously determined value of the control parameter does not always result in a reducing of the distension device. Similarly, a detected value of a control parameter that is less than a previously determined value of the control parameter does not always result in a expanding of the distension device.

In general, a control parameter can represent an operational parameter of the implantable distension system that can be directly controlled by a physician via adjustment of the adjustable distension device. Examples of control parameters include, but are not limited to, a pressure within the distension system, a peristaltic pulse event or frequency, a peristaltic pulse width, a peristaltic pulse duration, a peristaltic pulse amplitude, and a flow rate of a bolus into the stomach. A result parameter generally represents an output result of the implantable distension system that can be indirectly controlled by a physician via adjustment of the adjustable distension device. Examples of result parameters include, but are not limited to, the body mass index of the patient, the weight of the patient, the change in weight of the patient, and percent excess weight lost by the patient.

A detected value of the control parameter that is substantially equal to a pre-determined value of the control parameter can include variations in the detected value of the control parameter in the range of about 5-10%. A control parameter that substantially converges on a value over time can include variations in the value of the control parameter in the range of about 5-10%. Similar to the control parameter, a result parameter that substantially converges as a function of time can include variations in the value of the result parameter in the range of about 5-10%.

The present invention generally provides methods and devices for optimizing the operation of a distension system for causing distension in a stomach. In one exemplary embodiment, the method includes providing an implantable distension system for causing distension in a stomach, determining an optimum value of a control parameter of the distension system, and maintaining the control parameter at the optimum value such that a result parameter of the distension system is substantially convergent as a function of time. In one embodiment, determining an optimum value of a control parameter of the distension system can include adjusting the distension device, determining the value of a control parameter of the distension system, and repeating the steps of adjusting the distension device and determining the value of the control parameter until the control parameter is substantially convergent as a function of time.

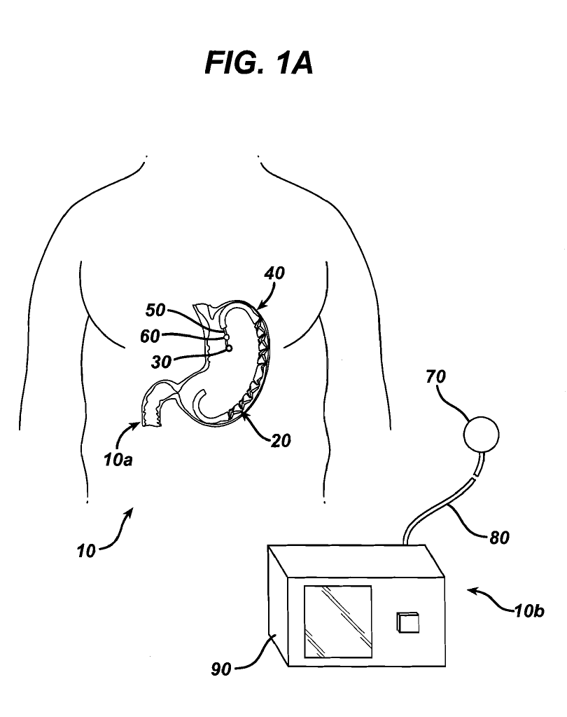

While the present invention can be used with a variety of distension systems known in the art, FIG. 1A illustrates one exemplary embodiment of a food intake distension system 10 in use in a patient. As shown, the system 10 generally includes an implantable portion 10a and an external portion 10b. FIG. 1B illustrates the implantable portion 10a outside of a patient. As shown, the implantable portion 10a includes an adjustable gastric coil 20 that is configured to be positioned in a patient's stomach 40 and an injection port housing 30 that is fluidly coupled to the adjustable gastric coil 20, e.g., via a catheter 50. The injection port 30 is adapted to allow fluid to be introduced into and removed from the gastric coil 20 to thereby adjust the size of the coil 20 and thus the pressure applied to the stomach 40. The injection port 30 can thus be implanted at a location within the body that is accessible endoscopically. Typically, injection ports are positioned in stomach wall or on the gastric coil itself.

The internal portion 10a can also include a sensing or measuring device that is in fluid communication with the closed fluid circuit in the implantable portion 10a. In one embodiment, the sensing device is a pressure sensing device configured to measure the fluid pressure of the closed fluid circuit. While the pressure measuring device can have various configurations and can be positioned anywhere along the internal portion 10a, including within the injection port 30 and as described further below, in the illustrated embodiment the pressure measuring device is in the form of a pressure sensor that is disposed within a sensor housing 60 positioned adjacent to the injection port 30. The catheter 50 can include a first portion that is coupled between the gastric coil 20 and the pressure sensor housing 60 and a second portion that is coupled between the pressure sensor housing 60 and the injection port 30. While it is understood that the sensing device can be configured to obtain data relating to one or more relevant parameters, generally it will be described herein in a context of a pressure sensing device.

In addition to sensing pressure of fluid within the internal portion 10a as described herein, pressure of fluid within the esophagus and/or the stomach 40 can also be sensed using any suitable device, such as an endoscopic manometer. By way of non-limiting example, such fluid pressure measurements can be compared against measured pressure of fluid within the internal portion 10a before, during, and/or after adjustment of pressure within the internal portion 10a. Other suitable uses for measured pressure within the esophagus and/or the stomach 40 will be appreciated by those skilled in the art.

As further shown in FIG. 1A, the receiving portion 10b generally includes a data reading device 70 that is configured to be positioned endoscopically via the mouth or on the skin surface above the pressure sensor housing 60 (which can be implanted in the stomach) to non-invasively communicate with the pressure sensor housing 60 and thereby obtain pressure measurements. The data reading device 70 can optionally be electrically coupled (wirelessly or wired, as in this embodiment via an electrical cable assembly 80) to a control box 90 that can display the pressure measurements, other data obtained from the data reading device 70, and/or data alerts, as discussed further below. While shown in this example as located local to the patient, the control box 90 can be at a location local to or remote from the patient.

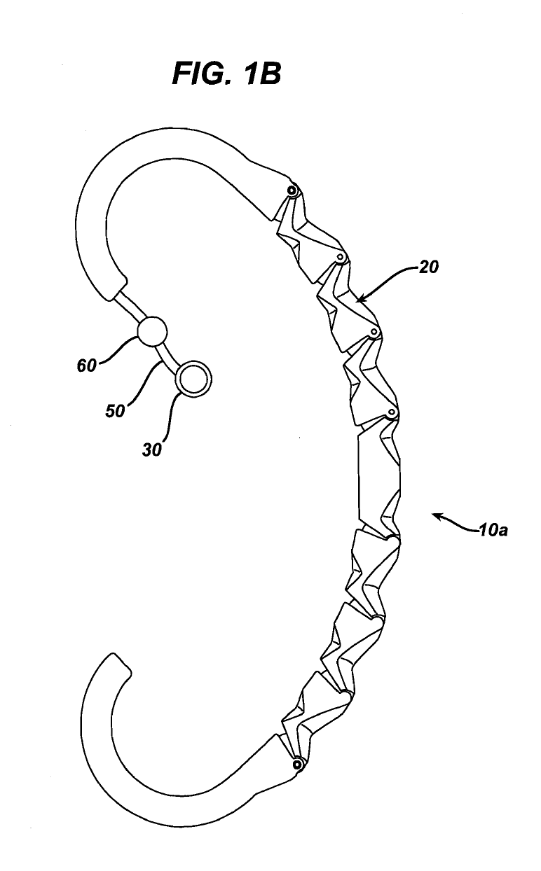

FIG. 2A shows the cross sectional view of the gastric coil 20 in more detail. While the gastric coil 20 can have a variety of configurations, and various gastric coils currently known in the art can be used with the present disclosure, in the illustrated embodiment the gastric coil 20 has a generally elongate shape with a support structure 22 having first and second opposite ends 20a, 20b that can be formed in a C-shape. Various techniques can be used to keep the ends 20a, 20b in relative proximity to one another. In the illustrated embodiment, the fluid bladder pressure may be varied to control the proximity of the ends relative to each other. The gastric coil 20 can also include a variable volume member, such as an inflatable balloon 24, that is disposed or formed on one side of the support structure 22 and that is configured to be positioned adjacent to tissue. The balloon 24 can expand or contract against the inner wall of support structure 22 to form an adjustable size coil for controllably restricting food intake into the stomach.

A person skilled in the art will appreciate that the gastric coil can have a variety of other configurations. Moreover, the various methods and devices disclosed herein have equal applicability to other types of implantable coils.

FIG. 2B shows the adjustable gastric coil 20 applied the stomach of a patient. As shown, the coil 20 at least substantially distends the stomach 40. After the coil 20 is implanted, it may be deployed. A person skilled in the art will appreciate that various techniques, including mechanical and electrical techniques, can be used to adjust the coil.

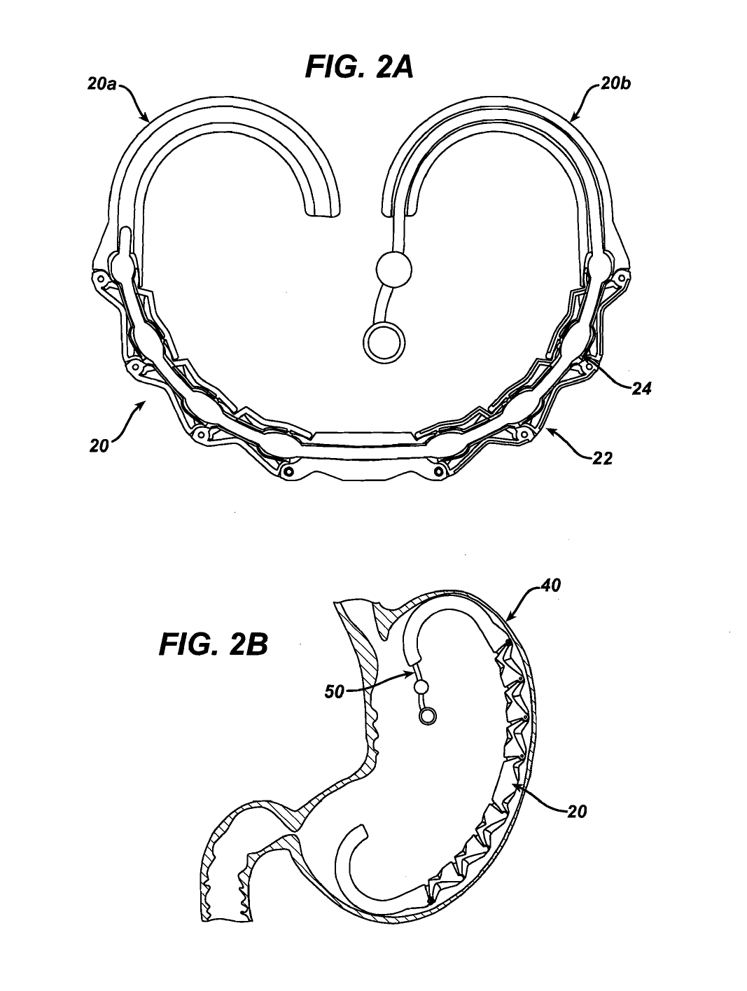

The fluid injection port 30 can also have a variety of configurations. In the embodiment shown in FIG. 3, the injection port 30 has a generally cylindrical housing with a distal or bottom surface and a perimeter wall extending proximally from the bottom surface and defining a proximal opening 32. The proximal opening 32 can include a needle-penetrable septum 34 extending there across and providing access to a fluid reservoir (not visible in FIG. 3) formed within the housing. The septum 34 is preferably placed in a proximal enough position such that the depth of the reservoir is sufficient enough to expose the open tip of a needle, such as an endoscopic Huber-like needle, so that fluid transfer can take place. The septum 34 is preferably arranged so that it will self seal after being punctured by a needle and the needle is withdrawn. As further shown in FIG. 3, the port 30 can further include a catheter tube connection member 36 that is in fluid communication with the reservoir and that is configured to couple to a catheter (e.g., the catheter 50). A person skilled in the art will appreciate that the housing can be made from any number of materials, including stainless steel, titanium, or polymeric materials, and the septum 34 can likewise be made from any number of materials, including silicone. An accumulator may be positioned between the fill port and bladder of gastric coil to provide constant pressure on the system. I.e. the coil may experience varying pressure due to gastric motility or motion, the accumulator will accommodate a reservoir of fluid at a constant pressure.

The reading device 70 can also have a variety of configurations, and one exemplary pressure reading device is disclosed in more detail in commonly-owned U.S. Publication No. 2006/0189888 and U.S. Publication No. 2006/0199997, which are hereby incorporated by reference. In general, the reading device 70 can non-invasively measure the pressure of the fluid within the implanted portion 10a. The physician can hold the reading device 70 against the patient's skin adjacent the location of the sensor housing 60 and/or other pressure sensing device location(s), obtain sensed pressure data and possibly other information as discussed herein, and observe the pressure reading (and/or other data) on a display on the control box 90. The data reading device 70 can also be removably attached to the patient, as discussed further below, such as during a prolonged examination, using straps, adhesives, and other well-known methods. The data reading device 70 can operate through conventional cloth or paper surgical drapes, and can also include a disposal cover (not shown) that may be replaced for each patient. Furthermore, the reading device may be operated using an endoscopic probe which may be inserted down the mouth of the patient to close proximity with the coil.

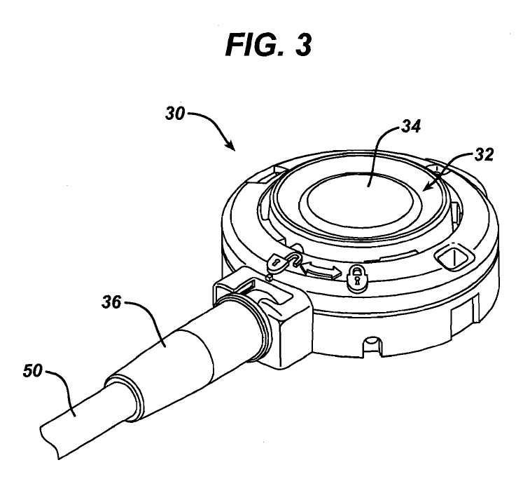

As indicated above, the system 10 can also include one or more sensors for monitoring the operation of the gastric distension system 10. The sensor(s) can be configured to measure various operational parameters of the system 10 including, but not limited to, a pressure within the system, a temperature within the system, a peristaltic pulse event or frequency, the peristaltic pulse width, the peristaltic pulse duration, and the peristaltic pulse amplitude. In one exemplary embodiment, the system can include a sensor in the form of a pressure measuring device that is in communication with the closed fluid circuit and that is configured to measure the fluid pressure within the system, which corresponds to the amount of distension applied by the adjustable gastric coil to the patient's stomach. As is explained below in detail, measuring the fluid pressure, or any other control parameter of the system, can enable a physician to evaluate the performance of the distension system. In the illustrated embodiment, shown in FIG. 4, the pressure measuring device is in the form of a pressure sensor 62 disposed within the sensor housing 60. The pressure measuring device can, however, be disposed anywhere within the closed hydraulic circuit of the implantable portion, and various exemplary locations and configurations are disclosed in more detail in commonly-owned U.S. Publication No. 2006/0211913 entitled “Non-Invasive Pressure Measurement In a Fluid Adjustable Restrictive Device,” filed on Mar. 7, 2006 and hereby incorporated by reference. In general, the illustrated sensor housing 60 includes an inlet 60a and an outlet 60b that are in fluid communication with the fluid in the implantable portion 10a. An already-implanted catheter 50 can be retrofitted with the sensor housing 60, such as by severing the catheter 50 and inserting barbed connectors (or any other connectors, such as clamps, clips, adhesives, welding, etc.) into the severed ends of the catheter 50. The sensor 62 can be disposed within the housing 60 and be configured to respond to fluid pressure changes within the hydraulic circuit and convert the pressure changes into a usable form of data.

Various pressure sensors known in the art can be used as the pressure sensor 62, such as a wireless pressure sensor provided by CardioMEMS, Inc. of Atlanta, Ga., though a suitable MEMS pressure sensor may be obtained from any other source, including but not limited to Integrated Sensing Systems, Inc. (ISSYS) of Ypsilanti, Mich. and Remon Medical Technologies, Inc. of Waltham, Mass. One exemplary MEMS pressure sensor is described in U.S. Pat. No. 6,855,115, the disclosure of which is incorporated by reference herein for illustrative purposes only. It will also be appreciated by a person skilled in the art that suitable pressure sensors can include, but are not limited to, capacitive, piezoresistive, silicon strain gauge, or ultrasonic (acoustic) pressure sensors, as well as various other devices capable of measuring pressure.

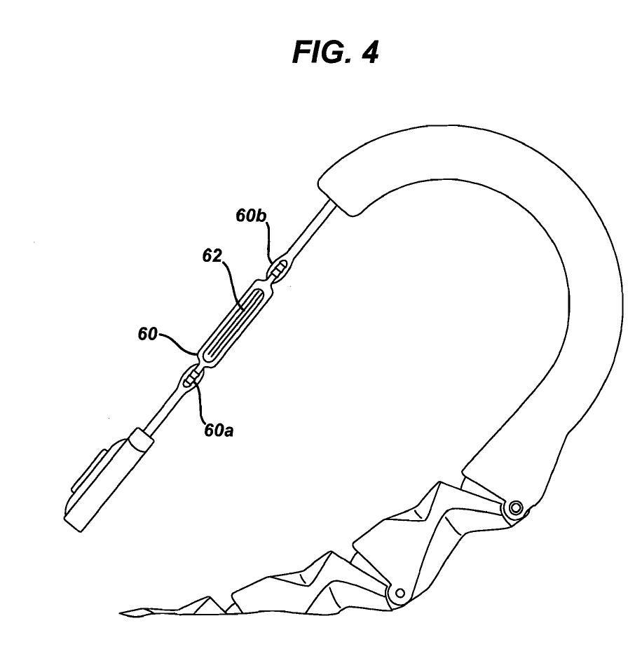

One embodiment of a configuration of the sensor housing 60 having the sensor 62 disposed within it is shown in FIG. 5. The sensor housing 60 in this example includes a motherboard that can serve as a hermetic container to prevent fluid from contacting any elements disposed within the sensor housing 60, except as discussed for the sensor 62. The sensor housing 60 can be made from any biocompatible material appropriate for use in a body, such as a polymer, biocompatible metal, and other similar types of material. Furthermore, the sensor housing 60 can be made from any one or more of transparent (as shown in FIG. 5), opaque, semi-opaque, and radio-opaque materials. A circuit board 64 including, among other elements, a microcontroller 65 (e.g., a processor), can also be disposed within the housing 60 to help process and communicate pressure measurements gathered by the sensor 62, and also possibly other data related to the coil 20. As further discussed below, the circuit board 64 can also include a transcutaneous energy transfer (TET)/telemetry coil and a capacitor. Optionally, a temperature sensor can be integrated into the circuit board 64. The microcontroller 65, the TET/telemetry coil, the capacitor, and/or the temperature sensor can be in communication via the circuit board 64 or via any other suitable component(s). The TET/telemetry coil and capacitor can collectively form a tuned tank circuit for receiving power from the external portion 10b and transmitting pressure measurements to a pressure reading device, e.g., the reading device 70. Moreover, to the extent that a telemetry component associated with the pressure sensor 62 is unable to reach a telemetry device external to the patient without some assistance, such assistance can be provided by any suitable number of relays (not shown) or other devices.

Fluid can enter the sensor housing 60 through an opening 66 located anywhere on the housing's surface (here, its bottom surface) and come into contact with a pressure sensing surface 68 of the sensor 62. The sensor 62 is typically hermetically sealed to the motherboard such that fluid entering the opening 66 cannot infiltrate and affect operation of the sensor 62 except at the pressure sensing surface 68. The sensor 62 can measure the pressure of fluid coming into contact with the pressure sensing surface 68 as fluid flows in and out of the opening 66. For example, the pressure sensing surface 68 can include a diaphragm having a deformable surface such that when fluid flows through the opening 66, the fluid impacts the surface of the diaphragm, causing the surface to mechanically displace. The mechanical displacement of the diaphragm can be converted to an electrical signal by a variable resistance circuit including a pair of variable resistance, silicon strain gauges. One strain gauge can be attached to a center portion of diaphragm to measure the displacement of the diaphragm, while the second, matched strain gauge can be attached near the outer edge of diaphragm. The strain gauges can be attached to the diaphragm with adhesives or can be diffused into the diaphragm structure. As fluid pressure within coil 20 fluctuates, the surface of the diaphragm can deform up or down, thereby producing a resistance change in the center strain gauge.

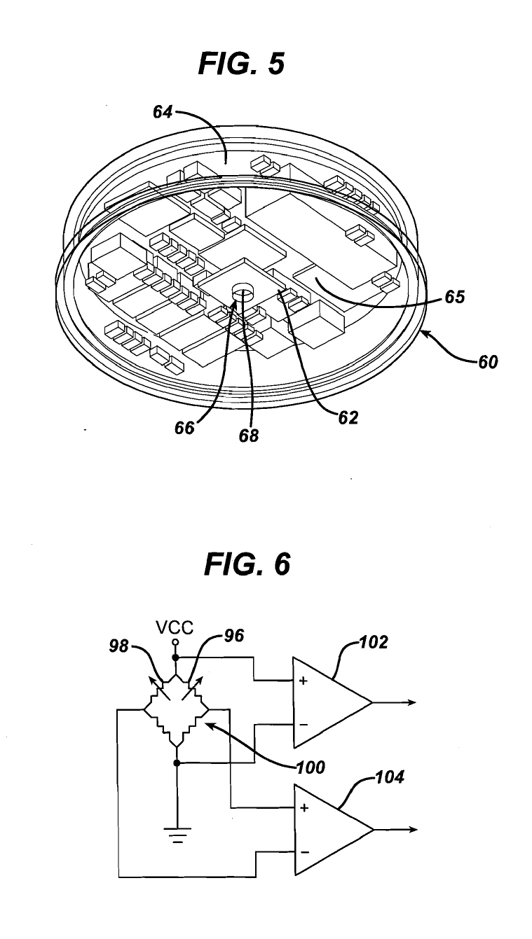

One embodiment of a variable resistance circuit for the sensor 62 is shown in FIG. 6. The circuit includes first and second strain gauges 96, 98 that form the top two resistance elements of a half-compensated, Wheatstone bridge circuit 100. As the first strain gauge 96 reacts to the mechanical displacements of the sensor's diaphragm, the changing resistance of the first gauge 96 changes the potential across the top portion of the bridge circuit 100. The second strain gauge 98 is matched to the first strain gauge 96 and athermalizes the Wheatstone bridge circuit 100. First and second differential amplifiers 102, 104 are connected to the bridge circuit 100 to measure the change in potential within the bridge circuit 100 due to the variable resistance strain gauges 96, 98. In particular, the first differential amplifier 102 measures the voltage across the entire bridge circuit 100, while the second differential amplifier 104 measures the differential voltage across the strain gauge half of bridge circuit 100. The greater the differential between the strain gauge voltages, for a fixed voltage across the bridge, the greater the pressure difference. Output signals from the differential amplifiers 102, 104 can be applied to the microcontroller 65 integrated into the circuit board 64, and the microcontroller 65 can transmit the measured pressure data to a device external to the patient. If desired, a fully compensated Wheatstone bridge circuit can also be used to increase the sensitivity and accuracy of the pressure sensor 62. In a fully compensated bridge circuit, four strain gauges are attached to the surface of diaphragm rather than only two strain gauges.

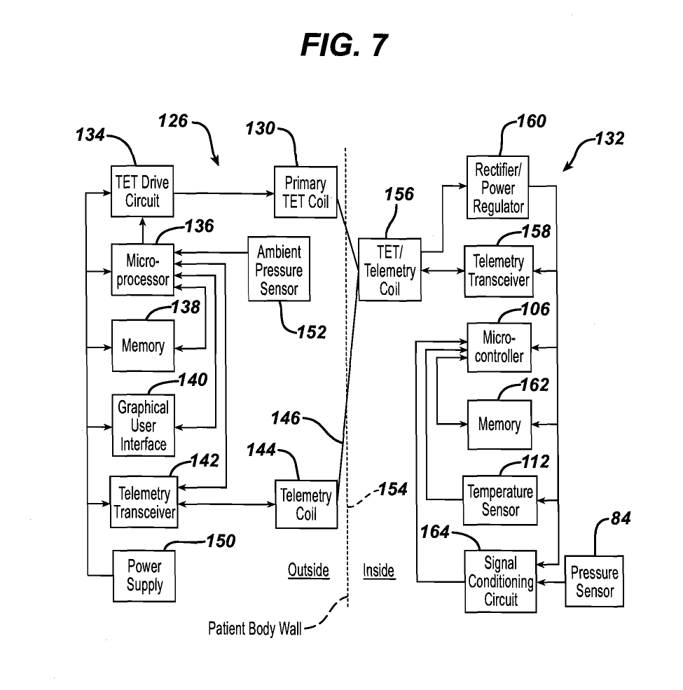

FIG. 7 illustrates one embodiment of components included in the internal and external portions 10a, 10b. As shown in FIG. 7, the external portion 10b includes a primary TET coil 130 for transmitting a power signal 132 to the internal portion 10a. A telemetry coil 144 is also included for transmitting data signals to the internal portion 10a. The primary TET coil 130 and the telemetry coil 144 combine to form an antenna, e.g., the reading device 70. The external portion 10b, e.g., disposed in the control box 90, includes a TET drive circuit 134 for controlling the application of power to the primary TET coil 130. The TET drive circuit 134 is controlled by a microprocessor 136 having an associated memory 138. A graphical user interface 140 is connected to the microprocessor 136 for inputting patient information, displaying data and physician instructions, and/or printing data and physician instructions. Through the user interface 140, a user such as the patient or a clinician can transmit an adjustment request to the physician and also enter reasons for the request. Additionally, the user interface 140 can enable the patient to read and respond to instructions from the physician and/or pressure measurement alerts.

The external portion 10b also includes a primary telemetry transceiver 142 for transmitting interrogation commands to and receiving response data, including sensed pressure data, from the implanted microcontroller 65. The primary transceiver 142 is electrically connected to the microprocessor 136 for inputting and receiving command and data signals. The primary transceiver 142 drives the telemetry coil 144 to resonate at a selected RF communication frequency. The resonating circuit can generate a downlink alternating magnetic field 146 that transmits command data to the microcontroller 65. Alternatively, the transceiver 142 can receive telemetry signals transmitted from a secondary TET/telemetry coil 114 in the internal portion 10a. The received data can be stored in the memory 138 associated with the microprocessor 136. A power supply 150 can supply energy to the control box 90 in order to power element(s) in the internal portion 10a. An ambient pressure sensor 152 is connected to microprocessor 136. The microprocessor 136 can use a signal from the ambient pressure sensor 152 to adjust the received pressure measurements for variations in atmospheric pressure due to, for example, variations in barometric conditions or altitude, in order to increase the accuracy of pressure measurements.

FIG. 7 also illustrates components of the internal portion 10a, which in this embodiment are included in the sensor housing 60 (e.g., on the circuit board 64). As shown in FIG. 7, the secondary TET/telemetry coil 114 receives the power/communication signal 132 from the external antenna. The secondary coil 114 forms a tuned tank circuit that is inductively coupled with either the primary TET coil 130 to power the implant or the primary telemetry coil 144 to receive and transmit data. A telemetry transceiver 158 controls data exchange with the secondary coil 114. Additionally, the internal portion 10a includes a rectifier/power regulator 160, the microcontroller 65, a memory 162 associated with the microcontroller 65, a temperature sensor 112, the pressure sensor 62, and a signal conditioning circuit 164. The implanted components can transmit pressure measurements (with or without adjustments due to temperature, etc.) from the sensor 62 to the control box 90 via the antenna (the primary TET coil 130 and the telemetry coil 144). Pressure measurements can be stored in the memory 138, adjusted for ambient pressure, shown on a display on the control box 90, and/or transmitted, possibly in real time, to a remote monitoring station at a location remote from the patient.

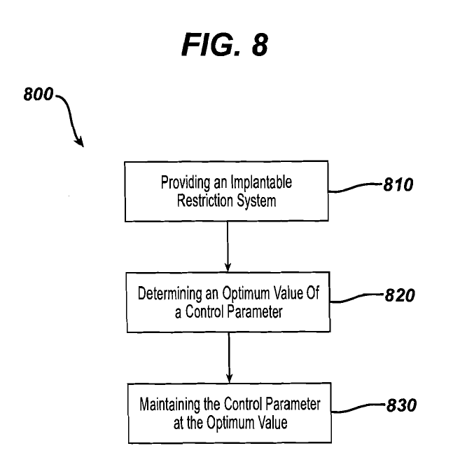

As indicated above, methods for optimizing the operation of a gastric distension system are disclosed herein. FIGS. 8-12 illustrate a variety of methods for optimizing the operation of the distension system 10. While the methods shown in FIGS. 8-12 are discussed with relation to the elements included in FIGS. 1A-7, a person skilled in the art will appreciate that the process can be modified to include more or fewer elements, reorganized or not, and can be performed in the distension system 10 disclosed herein or in another, similar system having other, similar elements.

FIG. 8 illustrates one exemplary embodiment of a method for optimizing the operation of a gastric distension system 800. The method can generally include providing an implantable distension system 810 for causing distension in a stomach, determining an optimum value of a control parameter 820 of the distension system, and maintaining the control parameter at the optimum value 830 such that a result parameter of the distension system is substantially convergent as a function of time. As indicated above, the implantable distension system of the method 800 can have a variety of configurations. In general, the distension system can include an adjustable distension device that is configured to cause distension in a stomach such as, for example, the gastric distension coil 20 described above. In one exemplary embodiment, the implantable distension system can take the form of the exemplary distension system 10 shown and described in FIGS. 1A-7.

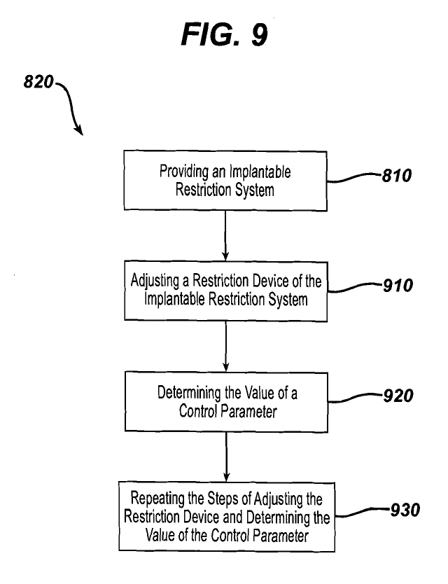

FIG. 9 illustrates one exemplary embodiment of a method for determining an optimum value of a control parameter 820. In general, a control parameter can represent an operational parameter of the implantable distension system that can be directly controlled by a physician via adjustment of the adjustable distension device. Examples of control parameters include, but are not limited to, a pressure within the distension system, flow rate of a bolus into the stomach, a peristaltic pulse event or frequency, a peristaltic pulse width, a peristaltic pulse duration, and a peristaltic pulse amplitude. Determining an optimum value of a control parameter 820 can generally include adjusting the distension device 910, determining the value of the control parameter to be optimized 920, and repeating the steps of adjusting the distension device and determining the value of the control parameter 930 until the control parameter is substantially convergent as a function of time.

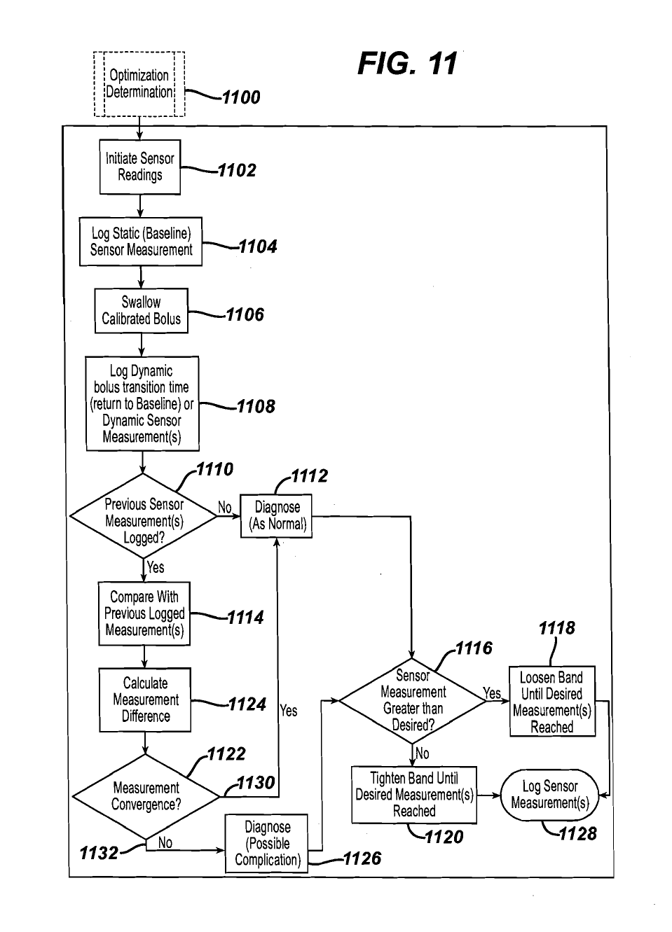

FIG. 11 illustrates the control parameter optimization process 1100 in greater detail. Once the physician has determined which control parameter is to be optimized, the optimization procedure can be initiated by taking an initial or baseline measurement of the control parameter 1102. One skilled in the art will appreciate that a variety of methods can be used to measure a value of the control parameter. For example, in one exemplary embodiment, a value of the control parameter can be detected using a sensor disposed in the distension system 10 such as, for example, the pressure sensor 62 described above. In particular, the pressure sensor 62 can be configured to count non-zero peristaltic pulses. After the baseline measurement is recorded 1104 by, for example, the microcontroller 65 discussed above, the patient can swallow a calibrated bolus 1106 to stimulate a peristaltic response. A dynamic sensor measurement can now be taken and recorded 1108 by the microcontroller 65. A dynamic sensor measurement can generally include, for example, measuring the value of the control parameter as the bolus enters the stomach.

As shown in FIG. 11, the determined value of the control parameter (i.e., the dynamic sensor measurement of the control parameter) can be compared to a previously determined value of the control parameter 1110 and the difference between the two values can be calculated 1124. If more than one dynamic sensor measurements are recorded for the patient, the most recently recorded measurement can be compared to the last recorded dynamic measurement for the control parameter 1114. If there is only one dynamic sensor measurement recorded for the patient, the recorded measurement can be compared to a pre-determined value for the control parameter 1112. In general, the pre-determined value can be a set number or range selected and known by the physician to correspond to the successful operation of a distension system in other patients. For example, in one exemplary embodiment, an experimentally pre-determined number of peristaltic pulses, such as 5-10 pulse counts, can serve as an initial baseline for acceptable number of peristaltic pulses indicating adequate system operation.

Regardless of whether the recorded dynamic measurement is compared to a previously recorded measurement 1114 or a pre-determined value for the control parameter 1112, the next step in the optimization procedure is the same. If the recorded dynamic sensor measurement and the previously determined value of the control parameter are substantially equal 1122, the system is operating at an optimum value 1130 and no adjustment of the distension device is necessary. However, if the recorded dynamic sensor measurement and the previously determined value of the control parameter are not substantially equal, this can indicate a possible complication with the operation of the system. Thus, if the measured value of the control parameter and the previously determined value of the control parameter are not substantially equal 1132, the physician can diagnose the possible complication 1126 and adjust the distension device to correct the complication. A number of factors can affect the adjustment of the coil. For example, the operating parameter chosen by the physician to be the control parameter, the measured value of the control parameter, and how the control parameter is measured can all influence the adjustment of the coil.

In one exemplary embodiment, the control parameter can be the peristaltic pulse duration and can be dynamically measured in seconds. If the recorded measurement of the peristaltic pulse duration is less than the previously determined value of the peristaltic pulse duration, the distension device can be expanded 1120. Expanding the coil can improve the performance of the system because a measured parameter that is less than the previously determined value generally corresponds to food passing too easily through the stomach. The distension device can generally be expanded by increasing the pressure within the distension system. In one embodiment, increasing the pressure within the distension system can include increasing the fluid pressure within the closed circuit of the system. In another embodiment, increasing the pressure within the distension system can include expanding the distension device itself (i.e., decreasing the diameter of the distension formed by the gastric coil as it is applied to the esophageal-gastric junction). As indicated above, several factors can affect the adjustment of the coil. Thus, a measured value of a control parameter that is less than a previously determined value of the control parameter does not always result in a expanding of the distension device. Exemplary embodiments of control parameters and measurement techniques that yield an expanding of the distension device when the measured value of the control parameter is less than the previously determined value of the control parameter include, but are not limited to, dynamic or static measurements of the pressure within the distension system in PSI or mmHg, dynamic measurements of the peristaltic pulse event by pulse count or pulse frequency, and dynamic measurements of the peristaltic pulse duration in seconds.

Alternatively, in another exemplary embodiment, if the recorded measurement of the control parameter is greater than the previously determined value of the control parameter 1116, the distension device can be reduced in size 1118. For example, in this embodiment, the control parameter can be the pressure within the distension system and can be statically or dynamically measured in either PSI or mmHg. Loosening the coil can improve the performance of the system because a measured parameter that is greater than the previously determined value generally corresponds to food either not passing or having difficulty passing through the distension at the esophageal-gastric junction. The distension device can be reduced in size by decreasing the pressure within the distension system. Decreasing the pressure within the distension system can include, for example, decreasing the fluid pressure within the closed circuit of the system and reducing the size of the distension device itself (i.e., increasing the diameter of the distension formed by the gastric coil as it is applied to stomach wall). As with the above embodiment, several factors can affect the adjustment of the coil. Thus, a measured value of the control parameter that is greater than a previously determined value of the control parameter does not always result in a reducing the size of the distension device.

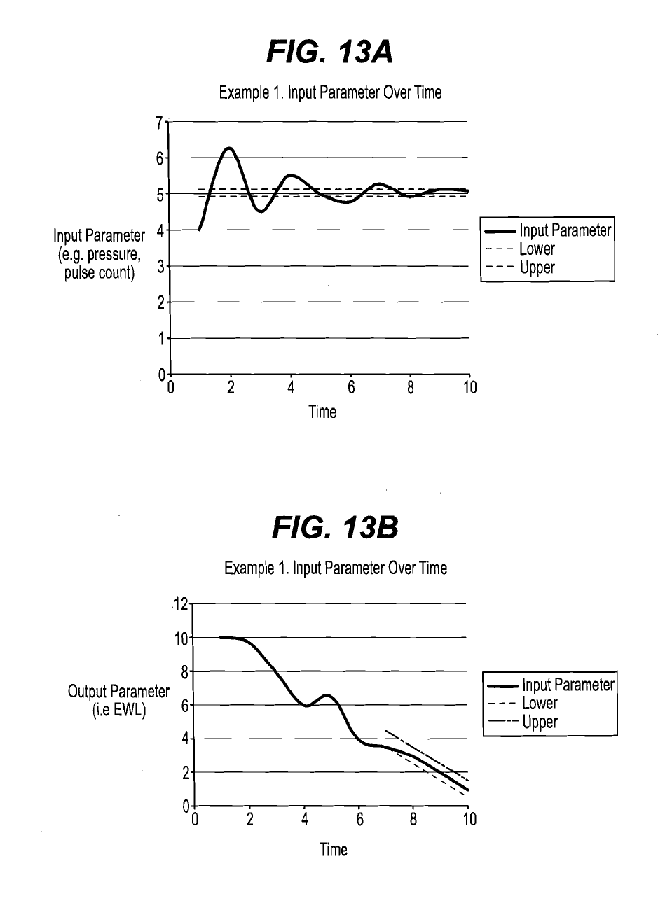

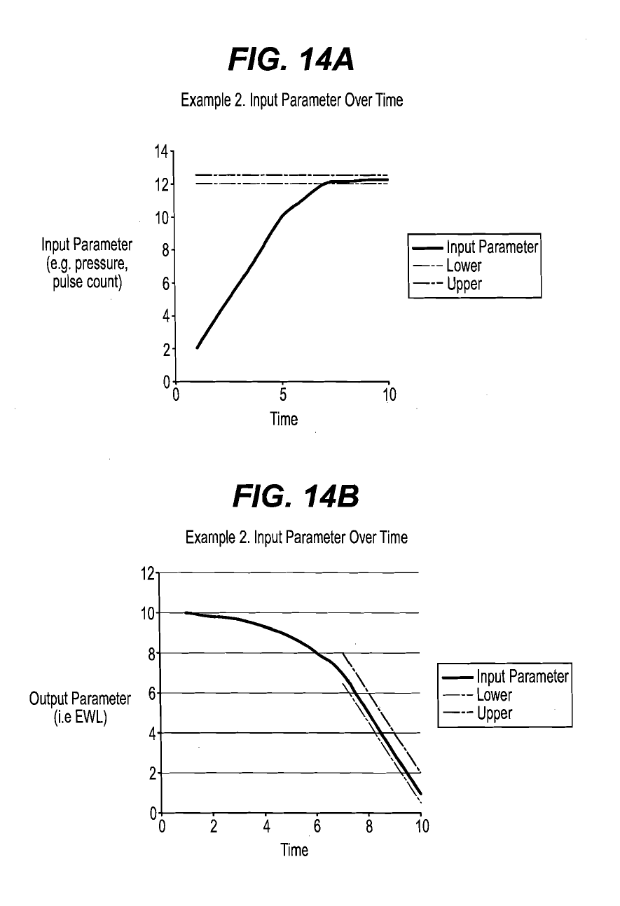

As indicated above, the steps of adjusting the distension device and determining the value of the control parameter can be repeated 930 until the control parameter is substantially convergent as a function of time (i.e., until the control parameter substantially converges on a value over time). For example, as shown in FIG. 11, the distension device can be adjusted 1118, 1120 until the determined value of the control parameter (i.e., the dynamic sensor measurement of the control parameter) and the previously determined value of the control parameter are substantially equal. The sensor measurements can be logged 1128 to create an optimization history for the patient. FIGS. 13A and 14A illustrate that maintaining the control parameter at a value that is substantially equal to a previously determined value of the control parameter over time can correspond to a control parameter that is substantially convergent as a function time. It is this value of the control parameter (i.e., the value of the control parameter that has substantially converged on a value over time) that can correspond to the optimum value of the control parameter for a specific patient. The terms substantially equal and substantially convergent or converges can include variations in the value of the control parameter in the range of about 5-10%.

Referring back to FIG. 8, once an optimum value of a control parameter of the distension system is determined 820, maintaining the control parameter at the optimum value 830 can be effective to substantially converge a result parameter of the distension system as a function of time. In general, a result parameter can represent an output result of the implantable distension system that can be indirectly controlled by a physician via adjustment of the adjustable distension device. Examples of result parameters include, but are not limited to, the body mass index of the patient, the weight of the patient, the change in weight of the patient, and percent excess weight lost by the patient. As shown in FIGS. 13A-14B, substantially maintaining the control parameter at an optimum value over time corresponds to a substantially convergent result parameter as a function of time. As with the control parameter, substantially convergent can include variations in the value of the result parameter in the range of about 5-10%. Substantially converging the result parameter as a function of time can be effective to optimize the operation of the distension system, as a substantially convergent result parameter generally corresponds with steady, consistent weight loss by the patient over time.

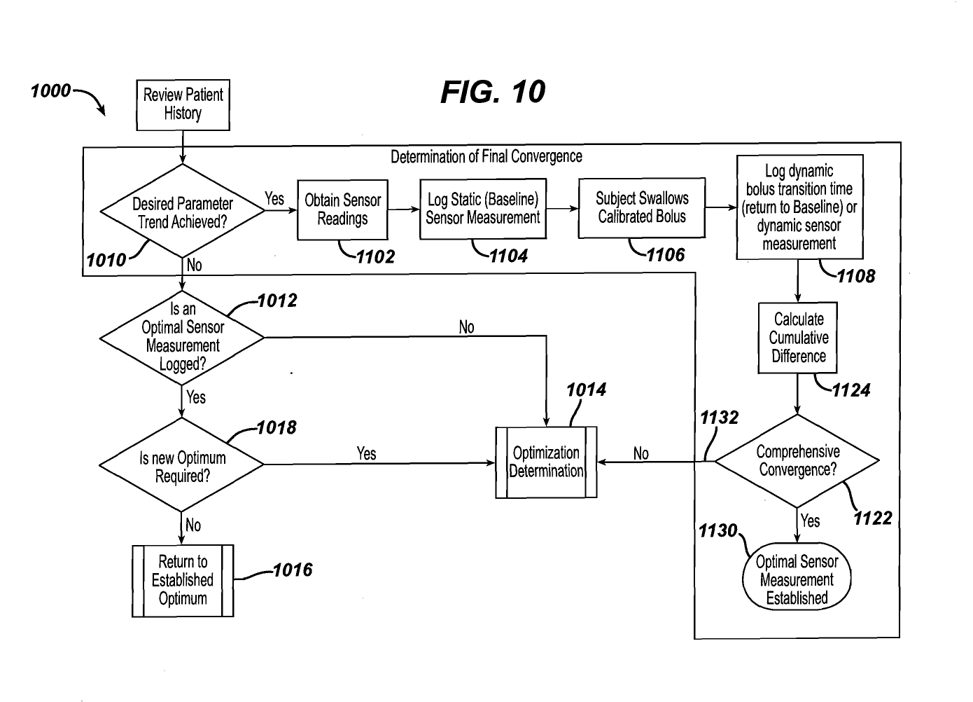

FIG. 10 cumulatively illustrates one exemplary embodiment of a method for optimizing the operation of a distension system 1000. As shown, the first step is to determine if the desired result parameter trend is achieved 1010 (i.e., is the patient losing weight at a steady, consistent rate?). If yes, the value of a control parameter can be substantially maintained by repeating the steps of measuring the control parameter and comparing the measured value to a pre-determined value for the control parameter as described above and shown in FIGS. 9 and 11. If the desired results parameter trend is not achieved (i.e., the patient is not losing weight at a steady, consistent rate), a determination can be made as to the optimum value of a control parameter of the system 1012. If an optimum value for a control parameter has not been determined, an optimum value for the control parameter can be determined 1014 as described above and shown in FIGS. 9 and 11. If an optimum value for a control parameter has already been determined and recorded, the control parameter can be returned to its established optimum value 1016.

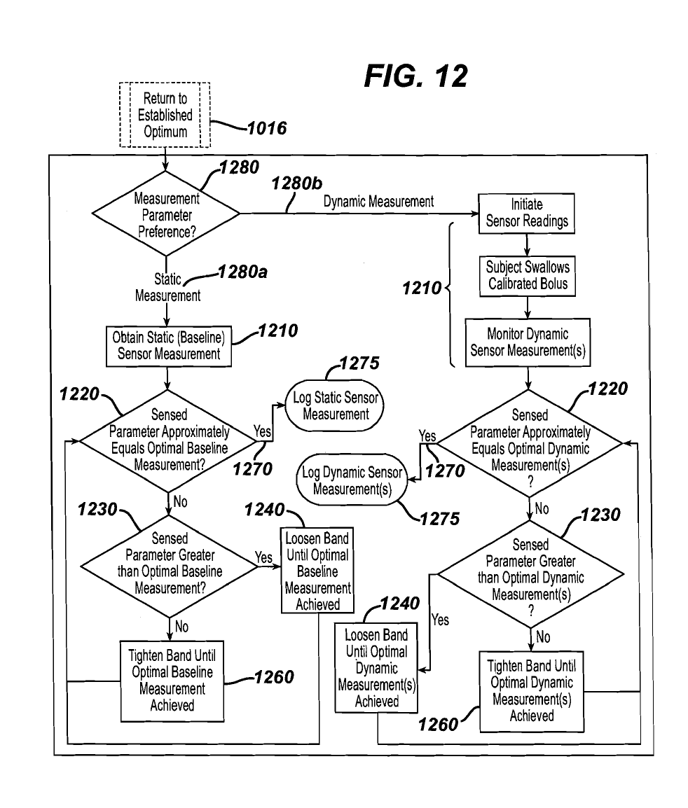

Returning the control parameter to a previously determined optimum value 1016 generally includes the steps of determining the current value of the control parameter 1210, comparing the current value of the control parameter to the previously determined optimum value for the control parameter 1220, and adjusting the distension device accordingly. As shown in FIG. 12, the physician can first select a measurement preference 1280. For example, the physician can chose to measure the current value of the control parameter statically 1280a or dynamically 1280b. As indicated above, a dynamic measurement of the control parameter can include, for example, measuring the value of the control parameter as a bolus enters the stomach. A static measurement of the control parameter can generally include measuring the “resting” value of the control parameter. For example, a static measurement of the control parameter can be taken in between mealtimes. Regardless of whether the current value of the control parameter is determined via static 1280a or dynamic 1280b measurement, the next step is to compare the measured value of the control parameter to the previously determined optimum value for the control parameter 1220. FIG. 12 illustrates three possible outcomes of the comparison 1220. In particular, if the measured value 1210 of the control parameter is substantially equal to the previously determined optimum value 1270, the control parameter is currently at its optimum value and the measured value 1210 can simply be logged 1275 as no adjustment of the distension device is necessary. If the measured value 1210 of the control parameter is greater than the previously determined optimum value 1230, the distension device can be reduced in size 1240 as described above with reference to FIG. 11. Alternatively, if the measured value 1210 of the control parameter is less than the optimum value 1250, the distension device can be expanded 1260 as described above with reference to FIG. 11. The steps of determining the current value of the control parameter 1210, comparing the current value of the control parameter to the previously determined optimum value of the control parameter 1220, and adjusting the distension device 1240, 1260 can be repeated until the measured value of the control parameter 1210 is substantially equal 1270 to the previously determined optimum value for the control parameter as such a comparison indicates that the control parameter has been returned to its established optimum value 1016. As noted above, the term substantially equal can include variations in the value of the control parameter in the range of about 5-10%.

Referring back to FIG. 10, in some embodiments, it may be necessary to determine a new optimum value for the control parameter 1018. For example, if a previously determined optimum value no longer corresponds with a desired result parameter trend, a new optimum value for the control parameter may need to be determined using the steps described above and shown in FIGS. 9 and 11.

It is understood that if a divergent data point is collected, and is attributed to a non representative event associated with measurement collection, such as wretching, vomiting, or other events, the data point collected may be discarded either manually or automatically.

In general, the methods disclosed herein for optimizing the operation of a distension system can minimize the guesswork required by a physician for a successful distension operation. Once an optimum value for a control parameter is determined, the physician or other person performing the system adjustments can input the same value each time without undue experimentation. Maintaining the optimum value for the control parameter can result in a convergent result parameter thereby yielding a distension system that produces predictable weight loss. This transforms system adjustment into a repeatable process that can be performed by less skilled personnel or by an automatically adjustable distension device.

Any patent, publication, application or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method for determining an optimum control parameter of a distension system for causing distension in a stomach, comprising:
a. providing a distension device having an undeployed shape for delivery within a hollow body and one or more deployed shapes for implantation therein, said member having sufficient rigidity in its deployed shape to exert an outward force against an interior of the hollow body so as to bring together two substantially opposing surfaces of said hollow body, and a means for changing the deployed shape of said member while implanted within said hollow body.
b. implanting the distension device within a body;
c. changing the shape of distension device to a first deployed shape;
d. determining the value of a control parameter of the distension system; and
e. changing the shape of distension device to one or more second deployed shapes until the control parameter is substantially convergent as a function of time.

2. The method of claim 1, further comprising comparing the determined value of the control parameter to a previously determined value of the control parameter.

3. The method of claim 2, wherein further comprising the step changing the shape of distension device if the determined value of the control parameter is not substantially equal to the previously determined value of the control parameter.

4. The method of claim 2, wherein changing the shape of distension device includes expanding the distension device if the determined value of the control parameter is less than the previously determined value of the control parameter.

5. The method of claim 2, wherein changing the shape of distension device includes reducing in size the distension device if the determined value of the control parameter is greater than the previously determined value of the control parameter.

6. The method of claim 1, wherein the step of determining the value of a control parameter of the distension system comprises measuring the pressure within the distension device.

7. The method of claim 1, wherein the wherein the step of determining the value of a control parameter of the distension system comprises measuring at least one of the following:
peristaltic pulse event, the peristaltic pulse width, the peristaltic pulse duration, number of peristaltic pulses, the peristaltic pulse amplitude, the flow rate of a bolus into the stomach.

8. A method for determining an optimum control parameter of a distension system for causing distension in a stomach, comprising:
a. providing a distension device having an undeployed shape for delivery within a hollow body and one or more deployed shapes for implantation therein, said member having sufficient rigidity in its deployed shape to exert an outward force against an interior of the hollow body so as to bring together two substantially opposing surfaces of said hollow body, and a means for changing the deployed shape of said member while implanted within said hollow body.
b. implanting the distension device within a body;
c. changing the shape of distension device to a first deployed shape;
d. determining an optimum value of a control parameter of the distension system; and
f. changing the shape of distension device to one or more second deployed shapes and maintaining the control parameter at the optimum value such that the parameter is substantially convergent as a function of time.

9. The method of claim 8, wherein the step of determining an optimum value of a control parameter of the distension system comprises detecting a value of the control parameter and comparing the detected value to a previously determined value of the control parameter.

10. The method of claim 9, wherein the step of changing the shape of distension device to one or more second deployed shapes comprises expanding the distension device if the detected value of the control parameter is less than the previously determined value of the control parameter.

11. The method of claim 9, wherein the step of changing the shape of distension device to one or more second deployed shapes comprises reducing in size the distension device if the detected value of the control parameter is greater than the previously determined value of the control parameter.

12. The method of claim 8, wherein substantially convergent includes variations in the value of the control parameter in the range of about 5-10% over time.

13. The method of claim 8, wherein substantially convergent includes variations in the value of the result parameter in the range of about 5-10% over time.

14. The method of claim 8, wherein the control parameter is the pressure within the distension system.

15. The method of claim 8, wherein the step of determining an optimum value of a control parameter of the distension system comprises measuring at least one of the following:
peristaltic pulse event, the peristaltic pulse width, the peristaltic pulse duration, the peristaltic pulse amplitude, number of peristaltic pulses and the flow rate of a bolus into the stomach.

16. The method of claim 8, wherein the step of determining an optimum value of a control parameter of the distension system comprises measuring least one of the following:
body mass index of the patient, the weight of the patient, number or duration of stomach pH excursions per day, the weight change of the patient, and the percent excess weight lost by the patient.
